(19) United States
(12) Patent Application Publication
(10) Pub. No.: US 2008/0206891 A1
(43) Pub. Date: Aug. 28, 2008

(54) Synthetic antiferromagnetic nanoparticles

(76) Inventors: Shan X. Wang, Portola Valley, CA (US); Robert John Wilson, Campbell, CA (US); Wei Hu, Stanford, CA (US)

Correspondence Address: LUMEN PATENT FIRM, INC., 2345 YALE STREET, SECOND FLOOR, PALO ALTO, CA, 94306, US

(21) Appl. No.: 11/894,046

(22) Filed: Aug. 16, 2007

Related U.S. Application Data
(63) Continuation-in-part of application No. 11/655,561, filed on Jan. 18, 2007, now abandoned.
Application No. 11/655,561 is a continuation-in-part of application No. 10/829,505, filed on Apr. 22, 2004.
(60) Provisional application No. 60/760,221, filed on Jan. 18, 2006.
(60) Provisional application No. 60/519,378, filed on Nov. 12, 2003.

(51) Int. Cl. G01N 33/553

(52) U.S. Cl. 436/526

(57) ABSTRACT

The present invention provides a synthetic antiferromagnetic (SAF) nanoparticle. The SAF nanoparticle includes at least two ferromagnetic layers and at least one non-magnetic spacer layer. The spacer layer is situated in between planar surfaces of the ferromagnetic layers. The saturation field of the SAF nanoparticle is tunable by the geometry and composition of the nanoparticle. Preferably, the saturation field can be tuned to be between about 100 Oe and about 10,000 Oe. Also preferably, the SAF nanoparticle has a magnetic moment of at least 800 emu/cm³. In a preferred embodiment, the SAF nanoparticle has at least one of a biomolecule, a recognition moiety, or a molecular coating attached to its surface. The SAF nanoparticle may also have a dye attached to its surface.

### CROSS-REFERENCE TO RELATED APPLICATIONS

This application is a continuation-in-part of U.S. patent application Ser. No. 11/655,561, filed Jan. 18, 2007, which claims priority from U.S. Provisional Patent Application No. 60/760,221, filed Jan. 18, 2006, both of which are incorporated herein by reference. U.S. patent application Ser. No. 11/655,561 is a continuation-in-part of U.S. patent application Ser. No. 10/829,505, filed Apr. 22, 2004, which claims priority from U.S. Provisional Patent Application No. 60/519,378, filed Nov. 12, 2003, all of which are incorporated herein by reference.

### STATEMENT REGARDING FEDERALLY SPONSORED RESEARCH OR DEVELOPMENT

This invention was supported in part by grant number N00014-02-1-0807 from the U.S. Navy and Defense Advanced Research Projects Agency (DARPA) and by grant number 1U54CA119367-01 from the National Cancer Institute. The U.S. Government has certain rights in the invention.

### FIELD OF THE INVENTION

The present invention relates generally to detection of agents. More particularly, the present invention relates to synthetic antiferromagnetic nanoparticles.

### BACKGROUND

Chemically synthesized superparamagnetic nanoparticles are widely used in biology and medicine for applications which include biomolecule purifications and cell separations, magnetic resonance imaging (MRI) contrast agents, and bio-magnetic sensing. Magnetic nanoparticles with higher moments are often desired to produce large signals or to avoid restrictive requirements for high magnetic field gradients in separations. Increasing the size of single grain superparamagnetic particles is not a viable route because these particles become coercive, and consequently spontaneously aggregate, at sizes above the superparamagnetic limit (˜12 nm for Fe). One solution is to incorporate numerous magnetic nanoparticles into larger composites using matrices comprised of dextran or silica. However, there are still limitations associated with controlling the monodispersity, magnetic response and variations in the number and size of the embedded nanoparticles. Accordingly, there is a need in the art to develop magnetic nanoparticles that overcome the above disadvantages.

### SUMMARY OF THE INVENTION

The present invention provides such magnetic nanoparticles. Specifically, the present invention provides a synthetic antiferromagnetic (SAF) nanoparticle. The SAF nanoparticle includes at least two ferromagnetic layers and at least one non-magnetic spacer layer. The spacer layer is situated in between planar surfaces of the ferromagnetic layers. The saturation field of the SAF nanoparticle is tunable by the geometry and composition of the nanoparticle. Preferably, the saturation field can be tuned to be between about 100 Oe and about 10,000 Oe. Also preferably, the SAF nanoparticle has a magnetic moment of at least 800 emu/cm³. In a preferred embodiment, the SAF nanoparticle has at least one of a biomolecule, a recognition moiety, and/or a molecular coating attached to its surface. The SAF nanoparticle may also have a dye attached to its surface.

The SAF nanoparticle may have additional layers in addition to ferromagnetic layers and spacer layers. Preferably, the SAF nanoparticle also includes a seed layer and a cap layer. Also preferably, the SAF nanoparticle has a layer with tunable plasmonic properties, a ferromagnetic layer with relaxation properties suitable for magnetic resonance imaging and detection, or a radioactive layer.

In addition to individual SAF nanoparticles, the present invention provides monodisperse solutions of SAF nanoparticles. Preferably, the solution contains a mixture of at least two types of nanoparticles. In one embodiment, each type of nanoparticle has a distinct saturation field and a distinct biomolecule, recognition moiety, and/or molecular coating. In another embodiment, each type of nanoparticle has a distinct magnetic, optical, radioactive, or relaxation property and a distinct biomolecule, recognition moiety, and/or molecular coating.

### BRIEF DESCRIPTION OF THE FIGURES

The present invention together with its objectives and advantages will be understood by reading the following description in conjunction with the drawings, in which:

FIG. 1 shows a schematic of a synthetic antiferromagnetic nanoparticle according to the present invention.

FIG. 2 shows a schematic of a solution of two types of SAF nanoparticles according to the present invention.

FIGS. 3-4 shows examples of tailoring magnetic properties of SAF nanoparticles according to the present invention.

FIG. 5 shows examples of fluorescent SAF nanoparticles according to the present invention.

### DETAILED DESCRIPTION OF THE INVENTION

### Structure of Synthetic Antiferromagnetic Nanoparticles

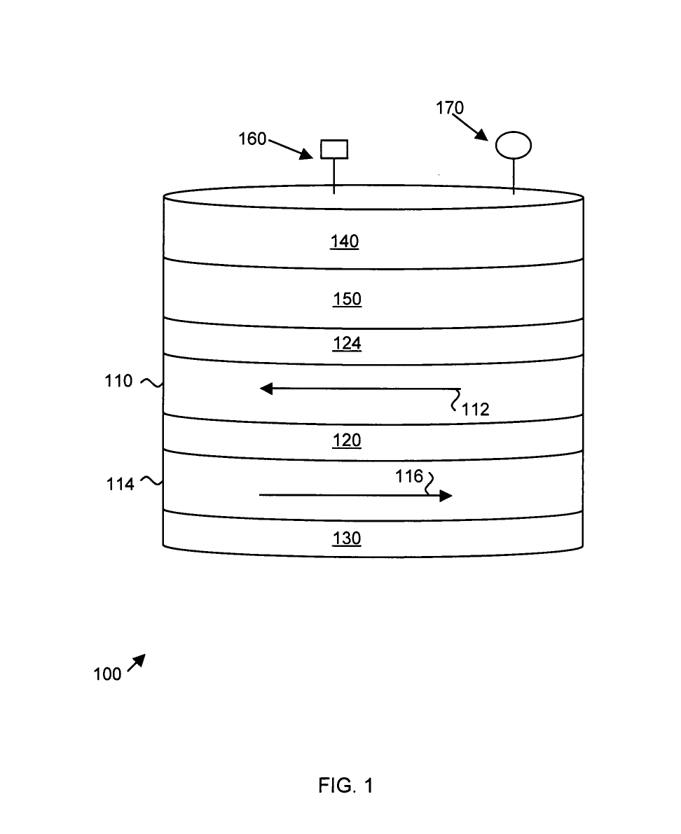

FIG. 1 shows a synthetic antiferromagnetic (SAF) nanoparticle 100 according to the present invention. SAF nanoparticle 100 includes ferromagnetic layers 110 and 114 separated by non-magnetic spacer layer 120. Ferromagnetic layers 110 and 114 are antiparallel, as indicated by arrows 112 and 116, respectively. SAF nanoparticle 100 also preferably includes non-magnetic spacer layer 124, seed layer 130, and cap layer 140. Also preferably included is layer 150, which conveys unique properties to the SAF nanoparticle. Layer 150 may, e.g., have tunable plasmonic properties, be a ferromagnetic layer with relaxation properties suitable for magnetic resonance imaging and detection, or be a radioactive layer. Alternatively, or in addition, SAF nanoparticle 100 may include dye 170 attached to its surface. SAF 100 nanoparticle also includes molecule 160 (or multiples of which) attached to its surface. Molecule 160 may be a biomolecule, a recognition moiety, and/or a molecular coating.

SAF nanoparticles according to the present invention have at least two ferromagnetic layers, although more may be used, such that the nanoparticles are made of “stacked units” of ferromagnetic layers separated by non-magnetic spacer layers. The ferromagnetic layers are preferably made of at least one of CoFe, Fe, Co, Ni, their alloys, or their oxides. Also preferably, two ferromagnetic layers have a combined total thickness of between about 10 nm and about 100 nm.

SAF nanoparticles according to the present invention also have at least one non-magnetic spacer layer, although more may be used as described above. The non-magnetic spacer layers are preferably made of at least one of ruthenium, gold, copper, tantalum, titanium, chromium, silicon nitride, or silicon dioxide. Preferably, each magnetic spacer layer is less than about 10 nm in thickness.

SAF nanoparticles according to the present invention also preferably have additional layers. For example, SAF nanoparticles preferably have at least one seed layer. The seed layer is preferably made of at least one of tantalum, ruthenium, chromium, or gold. SAFs also preferably have a cap layer, which is made of at least one of tantalum, chromium or gold. In addition, the nanoparticles may have layers that confer unique properties on it. Examples include, but are not limited to layers with tunable plasmonic properties, ferromagnetic layers with relaxation properties suitable for magnetic resonance imaging and detection, and radioactive layers. Alternatively, or in addition, a dye may be attached to a surface of the SAF nanoparticle. Preferably, this dye is fluorescent. Any fluorescent dye known in the art may be used.

SAFs according to the present invention also have at least one of a biomolecule, a recognition moiety, and/or a molecular coating attached to the surface of the nanoparticle. Examples of biomolecules include, but are not limited to, proteins, lipids, carbohydrates, peptides, nucleic acids, and oligonucleotides. Examples of recognition moieties include, but are not limited to, antibodies, oligonucleotides, and receptors. Examples of molecular coatings include, but are not limited to PEG or dextran polymers or various surfactants or charged molecules selected for colloidal solubility and stability.

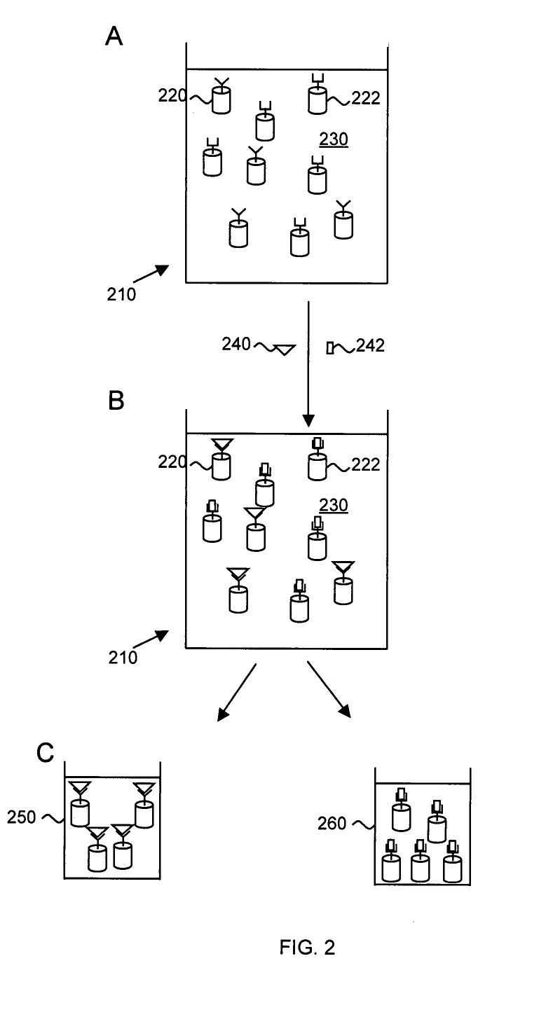

The present invention also provides solutions containing a plurality of SAF nanoparticles. The solution is preferably a monodisperse solution containing at least one type of SAF nanoparticle. More preferably, the solution contains a mixture of at least two types of SAF nanoparticles. In one embodiment, each type of SAF nanoparticle has a distinct saturation field value and a distinct biomolecule, recognition moiety, and/or molecular coating. In another embodiment, each type of SAF nanoparticle has a distinct magnetic, optical, radioactive, or relaxation property and a distinct biomolecule, recognition moiety, and/or molecular coating. In this way, different types of SAF nanoparticles can easily be distinguished and separated in the solution, thereby allowing different molecules, cells, etc. to be separated in the solution upon binding of the SAF nanoparticles to the molecules, cells, etc. in the solution. For example, FIG. 2A shows a container 210 with two types, 220 and 222, of SAF nanoparticles in solution 230. Upon addition of entities 240 and 242, SAF nanoparticle 220 recognizes entity 240, and SAF nanoparticle 222 recognizes entity 242 (FIG. 2B). SAF nanoparticles 220 and 222 can then be separated, along with their respective entities, into containers 250 and 260 (FIG. 2C).

### Fabrication of Synthetic Antiferromagnetic Nanoparticles

In one embodiment, the production of SAF nanoparticles relies on the fabrication of precise nanotemplates using Nanoimprint Lithography (NIL). A quartz stamp may be used, fabricated using electron beam lithography, which has 100 nm diameter pillars at 300 nm pitch in a square array. In another embodiment, inexpensive stamps may be produced using self-assembled polymer spheres. In this case, packed arrays of carboxylate-modified latex nanoparticles (CML) serve as etch masks for production of pillar arrays covering silicon wafers. The latex particle diameters may be reduced by etching with an oxygen containing plasma and this pattern may be transferred into Si pillars by etching with NF₃. The inventors have readily made Si nanopillars with this method having diameters of about 60 nm.

In either case, the imprinted templates are used as substrates for the deposition of thin multilayer magnetic films with precise thickness control. Preferably, release layers, resist bilayers, and metallization layers are deposited sequentially on the substrate. The resist layers and overlying metal film may then be removed using sonication in solvents, and the nanoparticles may then be released using liquid etches and surfactants to stabilize them in solution.

In one embodiment, proteins (such as streptavidin and antibodies), oligomers, and/or PEGs can be directly absorbed by the SAF nanoparticles. Alternatively, biomolecules, recognition moieties, and/or molecular coatings can be conjugated to SAF nanoparticles through gold-thiol linkage as widely practiced in biochemistry. In either case, these proteins, oligomers, and PEGs can incorporate dyes before or after being conjugated to the SAF nanoparticles.

### Magnetic Properties of Synthetic Antiferromagnetic Particles

SAF nanoparticles according to the present invention preferably have a saturation field that is tunable from about 100 Oe to about 10,000 Oe. Various factors contribute to this tunability, including the geometry and the composition of the SAF. In addition, SAFs preferably have magnetic moments per unit volume of at least 800 emu/cm³.

The magnetizations of adjacent ferromagnetic layers may be antiparallel in the absence of a magnetic field for a number of different reasons, including magnetostatic coupling, interfacial exchange coupling, and use of a coercive layer. For example, SAF nanoparticles can be made having a hard ferromagnetic layer (more coercive) and a soft ferromagnetic layer (less coercive), separated by a nonmagnetic spacer layer. The hard magnetic layer is magnetized to form a single magnetic domain, and the magnetostatic coupling can force the soft magnetic layer to form a single magnetic domain in the antiparallel configuration.

The magnetostatic interactions are primarily of two forms. One type is associated with shape anisotropies wherein the preferred directions for the magnetization are determined by the demagnetizing fields associated with non-spherical shapes of the nanoparticles. These interactions lead to in-plane easy axes for cylindrical features where the thickness of the magnetic layers is less than the nanoparticle diameter. As the nanoparticle shape deviates from cylindrical symmetry, specific in-plane axes (long axes usually) similarly become preferred. This can be highly valuable for locking or linking the magnetic axes of the nanoparticle to its physical axes, making the particle highly susceptible to in-plane rotational orientation by magnetic fields. In the simple case described here the nanoparticles are nominally cylindrically symmetric and the dominant anisotropy effect is preferential orientation of the particles so that the magnetization lies in the plane of the magnetic layers. The other type of magnetostatic interaction involves the relative orientation of the magnetic moments of the different magnetic layers within the nanoparticle, as depicted in FIG. 1, which illustrates that the magnetization in the two ferromagnetic layers are antiparallel to each other. This orientation is preferred due to the attraction of opposite magnetic poles and repulsion of like poles. These interactions can be quite strong and depend roughly on the aspect ratio t/D (t is the magnetic layer thickness and D is the diameter of the particle). This antiparallel orientation can be overcome by applying an external field. The saturation field of the nanoparticles can be tuned by adjusting the thickness of the ferromagnetic layers.

Interfacial magnetic coupling is a second useful method to control the magnetic characteristics of SAFs. One example is afforded by the use of thin layers of specific metals, notably Ru and Cr, which are sandwiched between ferromagnetic layers. This interfacial coupling has an oscillatory character as a function of the spacer layer thickness. It is manifested as a coupling energy, which can favor either antiparallel or parallel orientation of the adjacent magnetic layers. The resulting saturation fields also depend on magnetic layer thickness, although in a different manner than the magnetostatic interaction described above, and can also be exploited to tune the saturation field of un-patterned films. This antiferromagnetic interfacial coupling can be used to increase the saturation field of patterned multilayer samples to higher values than provided by magnetostatic coupling alone.

### EXAMPLES

### Direct Physical Fabrication of Synthetic Antiferromagnetic Nanoparticles

Nanoparticle fabrication began with vacuum coating of the substrate with a chemically etchable release layer of copper. A thin buffer layer of tantalum was also deposited to prevent oxidation of the Cu during subsequent resist bakes. All metal layers were deposited, at rates near 0.1 nm/sec, in a load locked vacuum system wherein a 1 keV, 10 mA xenon ion beam was directed at carousel-mounted targets at an operating pressure of 2×10⁻⁴ Torr. Next, a layer of polymethylglutarimide (PMGI) undercut resist (MicroChem) was spin coated onto the metal release layer and baked at 200° C. for 5 minutes. A layer of polymethyl methacrylate resist, PMMA, (MicroResistTechnology, 75 k MW), was then spin coated onto the wafer and baked at 140° C. for 5 minutes. The thickness of each resist layer was adjusted to accommodate stamp dimensions and particle thickness. The templates were then formed in the PMMA resist using thermal nanoimprinting at 40 bar for 60 s at 180° C., which is above the glass transition temperature of PMMA, T_{G}=105° C., but below T_{G}=200° C. for PMGI. The quartz stamp, with a patterned area of 1 cm² containing 10⁹ pillars which are 100 nm in diameter and 200 nm in height, and NIL tool were purchased from Obducat. After several minutes of cooling, the imprint and stamp were carefully separated using a mechanical wafer chuck and vacuum tweezers. A thin residual layer of PMMA was then removed by oxygen plasma treatment and a wet chemical developer, LDD-26W (Shipley), was used to generate an undercut lift-off profile by selectively and isotropically removing a portion of the PMGI resist. This produces an array of holes in PMMA resist, atop undercut holes in PMGI and a continuous release layer film. The patterned wafers were next returned to the vacuum deposition tool where multilayers were sequentially deposited. After lift-off, the final fabrication step was to release the particles by ion milling through the thin Ta buffer layer and then chemically etching the Cu release layer with an ammonia-CuSO₄ solution which exploits Cu-ammine complexes to attain high selectivity towards Cu. This etch was neutralized by the addition of citrate buffer, which also acts as a surfactant to stabilize the nanoparticles in solution. The particles were collected by multiple cycles of centrifugation, solvent exchange, and re-suspension.

When these particles are subjected to a magnetic field of ˜1 kOe and a field gradient of ˜1 kOe/cm, they yield a magnetically induced velocity of ˜3 μm/sec. The particle-to-particle variation in magnetic drift velocity is negligible, consistent with the monodispersity of the SAF nanoparticles. The saturation magnetization of SAF nanoparticles with 12 nm magnetic layer thickness is measured to be ˜850 emu/cm³.

### Tailoring Magnetic Properties of SAF Nanoparticles

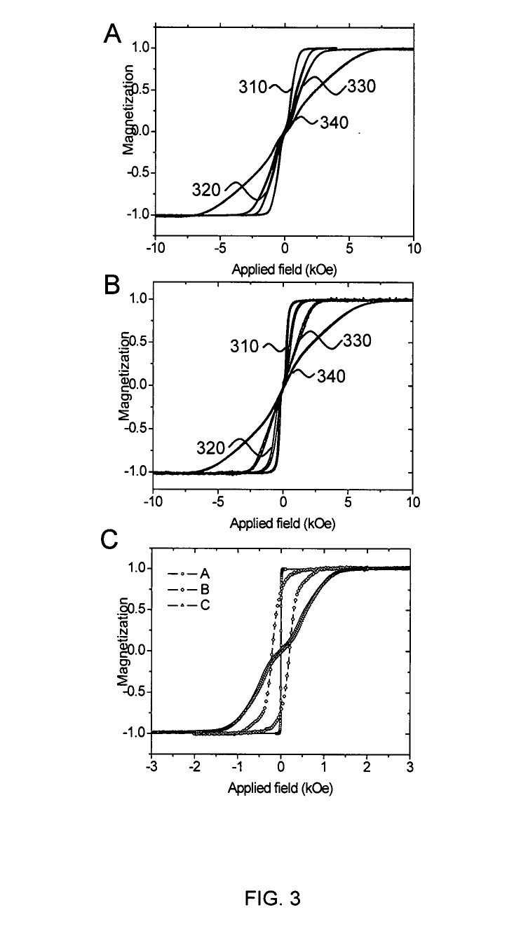

The magnetic properties of substrate-bound nanoparticles, as well as released nanoparticles in aqueous solution, have been measured by alternating gradient magnetometry (AGM). FIG. 3A shows hysteresis loops (normalized) of substrate-bound 120 nm diameter SAF nanoparticles comprised of stacks of Ta(5 nm)/Ru(2)/CoFe(t)/Ru(2.5)/CoFe(t)/Ru(2)/Ta(5) for magnetic bilayer thicknesses of t=6 nm (curve 310) and 12 nm (curve 320), resulting in a total thickness of 28.5 nm and 40.5 nm, respectively. The remanence and coercivity of these nanoparticles are nearly zero, dramatically reduced from those of single layer CoFe nanoparticles of the same size (FIG. 3C). The functional dependence of the magnetization M, is M(H)=M_{S}(H/H_{S}) until M attains a constant value M_{S} when H reaches the saturation field H_{S}. M_{S} and H_{S} are both proportional to the magnetic bilayer thickness because interlayer magnetic repulsion increases linearly with t, as expected from considerations of demagnetizing fields. Deviations from linearity at small fields are associated with as-deposited anisotropies which cause a non-zero “spin flop” field. SAF nanoparticles with identical diameters using only this thickness-dependent magnetostatic repulsion have identical moments at low fields, but their moments differ at high fields (after the onset of saturation).

The magnetic saturation fields can be further tailored by employing a special spacer between the magnetic layers that produces strong magnetic interfacial exchange coupling. This subtle quantum phenomenon depends very strongly on the non-magnetic spacer material, often ruthenium, and its thickness. The effects on hysteresis loops can be included by considering the interfacial exchange coupling as producing an effective magnetic field that adds to, or subtracts from, the magnetostatic demagnetizing field. This effect is quite pronounced for thin (<1 nm) ruthenium spacers, which provide strong antiferromagnetic coupling and thus increase the saturation field (curve 330 in FIG. 3A where the Ru spacer thickness is reduced to 0.6 nm). The saturation field can be further increased by increasing the number of interfaces and magnetic layers, while keeping the sum of magnetic layer thicknesses constant (curve 340 in FIG. 3A where 0.6 nm Ru spacers separate 3 nm CoFe layers). SAF nanoparticles exploiting variable interfacial exchange interactions can thus be made to have identical saturation moments at high fields, while their moments differ at low fields (before the onset of saturation).

FIG. 3B shows that the hysteresis loops of the magnetic nanoparticles of FIG. 3A, when released into solution, are modified, as is manifested by a reduction of saturation field. At the concentrations used for alternating gradient magnetometer (AGM) measurements (10¹⁰ particles/mL), these magnetic particles are observed to reversibly form field induced chain structures, induced by inter-particle interactions of magnetized nanoparticles, which are expected to have reduced saturation fields. The suspended SAF nanoparticle chains retain low remanence and even the distinctiveness of their saturation fields.

If Ru is used as a nonmagnetic spacer layer in the SAF nanoparticles, the remanence and coercivity of these nanoparticles are nearly zero (FIG. 3C, curve C: Ta(5)/Ru(2)/CoFe(6)/Ru(2.5)/CoFe(6)/Ru(2)/Ta(5) patterned, 120 nm). If these same particles are produced using a single magnetic layer, the coercivity and remanence are dramatically increased (FIG. 3C, curve B: Ta(5)/Ru(2)/CoFe(12)/Ru(2)/Ta(5) patterned, 120 nm), resulting in aggregation. These changes in magnetic properties are not simply associated with the magnetic material, but rather are consequences of patterning, as is evident in the low coercivity of continuous films (FIG. 3C, curve A: Ta(5)/Ru(2)/CoFe(12)/Ru(2)/Ta(5) continuous).

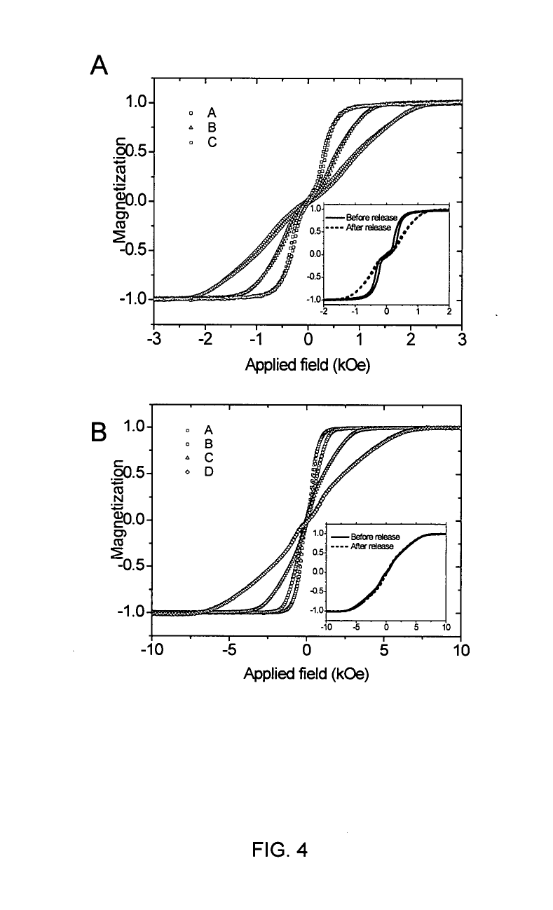

Additional examples of controlling magnetic properties with magnetostatic and interfacial (RKKY) exchange coupling are given in FIG. 4. FIG. 4A shows more detailed hysteresis loops (normalized) of substrate-bound 100 nm diameter nanoparticles made from stacks of Ta(5 nm)/Ru(1)/CoFe(t)/Ru(2.5)/CoFe(t)/Ru(1)/Ta(5)/Au(2) for magnetic bilayer thicknesses of t=3, 6 and 12 nm, respectively. The observed scaling of the saturation fields, which increase with magnetic layer thickness, is expected from simple considerations of demagnetizing fields. The inset in FIG. 4A shows that the hysteresis loops of the magnetic nanoparticles in solution are modified as is manifested by an apparent reduction of the saturation field. This reduction is attributed to magnetic chain formation, which is driven by inter-particle interactions of magnetized nanoparticles. The field produced by a single nanoparticle with a moment of m in contact with another, as will occur within a chain, is roughly m/D³=πMt/2D=H_{D}/2π, which causes the saturation field to decrease by a substantial fraction if the antiferromagnetic coupling in the SAF nanoparticles is mainly due to demagnetizing fields. Even with these interactions, the suspended SAF nanoparticle chains retain their low remanence and their saturation fields remain distinct.

The magnetic saturation fields are further tailored by employing a special Ru spacer between the magnetic layers that produces strong magnetic interfacial exchange coupling, as is shown in detail in FIG. 4B. The magnetic interactions between two magnetic films adjacent to this special spacer layer typically oscillate between antiferromagnetic and ferromagnetic and diminish in strength as the spacer layer thickness is increased from 0 to 3 nm. FIG. 4B shows the expected increase in saturation fields obtained by changing the Ru spacers from 2.5 nm (curve A) to 1.7 nm (curve B) to 0.6 nm (curve C), while keeping the two CoFe layers at a thickness of 6 nm each. Similarly, using more laminated CoFe layers with a thickness of 3 nm each (curve D) while keeping the Ru spacer at 0.6 nm greatly increased the saturation filed to ˜7 kOe. The inset shows the hysteresis loops of the magnetic nanoparticles (curve D) in solution before and after release from the substrate. It is worth noting that the reduction of saturation field due to inter-particle interactions is less apparent for SAF nanoparticles with stronger antiferromagnetic interfacial exchange coupling. This is to be expected since the maximum strength of inter-particle interactions depends upon the particle magnetic moments, and not on interfacial exchange coupling.

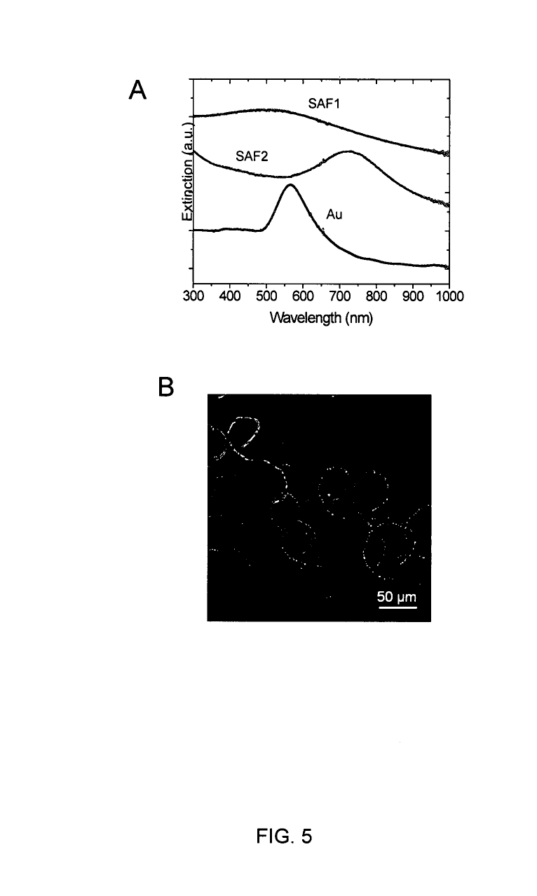

The NIL-based fabrication of the SAF nanoparticles not only provides desired tunability of the magnetic properties, but also allows customized incorporation of materials with unique properties. For example, an optional Au layer can be deposited on the top or bottom of the cap layer during the fabrication process, resulting in nanoparticles with a localized surface plasmon band (curve SAF2 in FIG. 5A). This band, positioned at 730 nm, falls within the range of surface plasmon wavelengths reported for Au nanodisks at similar sizes. This near infrared (NIR) wavelength is more desirable than that of spherical Au nanoparticles (bottom curve of FIG. 5A) for surface plasmon biosensors and applications such as photo-thermal cancer therapy. Without Au capping, the UV-Vis-NIR spectrum of the SAF nanoparticles shows a broad band in the visible region (curve SAF1 of FIG. 5A). Fluorescent nanoparticles are obtained via surface attachment of dyes. FIG. 5B shows the paths of AlexaFluor 594 labeled SAF nanoparticle clusters integrated over the course of a rotation of a magnetic field gradient, demonstrating magnetic and fluorescent multi-functionality.

As one of ordinary skill in the art will appreciate, various changes, substitutions, and alterations could be made or otherwise implemented without departing from the principles of the present invention. For example, the gold layer in the particles demonstrated in FIG. 5A could be replaced by a radioactive layer. Accordingly, the scope of the invention should be determined by the following claims and their legal equivalents.

What is claimed is:

1. A synthetic antiferromagnetic nanoparticle, comprising:
a) at least two ferromagnetic layers;
b) at least one non-magnetic spacer layer, wherein said at least one non-magnetic spacer layer is situated in between planar surfaces of said at least two ferromagnetic layers,
wherein the saturation field of said antiferromagnetic nanoparticle is tunable from about 100 Oe to about 10,000 Oe by the geometry and composition of said nanoparticle;
wherein said synthetic antiferromagnetic nanoparticle has a saturation magnetic moment per unit volume of at least 800 emu/cm³; and
wherein said synthetic antiferromagnetic nanoparticle comprises at least one of a biomolecule, a recognition moiety, or a molecular coating attached to a surface of said nanoparticle.

2. The synthetic antiferromagnetic nanoparticle as set forth in claim 1, wherein
magnetizations of adjacent ferromagnetic layers are antiparallel due to at least one of magnetostatic coupling, use of a coercive layer, or interfacial exchange coupling in the absence of applied magnetic field.

3. The synthetic antiferromagnetic nanoparticle as set forth in claim 1, wherein said at least two ferromagnetic layers comprise at least one of CoFe, Fe, Co, Ni, and their alloys or oxides.

4. The synthetic antiferromagnetic nanoparticle as set forth in claim 1, wherein said at least two ferromagnetic layers have a combined total thickness of between about 10 nm and 100 nm.

5. The synthetic antiferromagnetic nanoparticle as set forth in claim 1, wherein said non-magnetic spacer layer comprises ruthenium, gold, copper, tantalum, titanium, chromium, silicon nitride or silicon dioxide.

6. The synthetic antiferromagnetic nanoparticle as set forth in claim 1, wherein said non-magnetic spacer layer is less than about 10 nm in thickness.

7. The synthetic antiferromagnetic nanoparticle as set forth in claim 1, further comprising at least one seed layer, wherein said seed layer comprises at least one of tantalum, ruthenium, chromium or gold.

8. The synthetic antiferromagnetic nanoparticle as set forth in claim 1, further comprising a cap layer, wherein said cap layer comprises at least one of tantalum, ruthenium, chromium or gold.

9. A solution comprising a plurality of synthetic antiferromagnetic nanoparticles as set forth in claim 1, wherein said solution contains a mixture of at least two types of said synthetic antiferromagnetic nanoparticles, wherein each of said types has a distinct saturation field value and a distinct biomolecule, recognition moiety, molecular coating, or combination thereof.

10. The synthetic antiferromagnetic nanoparticle as set forth in claim 1, further comprising at least one layer that has tunable plasmonic properties.

11. A solution comprising a plurality of synthetic antiferromagnetic nanoparticles as set forth in claim 10, wherein said solution contains a mixture of at least two types of said synthetic antiferromagnetic nanoparticles, wherein each of said types has a distinct plasmonic property and a distinct biomolecule, recognition moiety, molecular coating, or combination thereof.

12. The synthetic antiferromagnetic nanoparticle as set forth in claim 1, further comprising at least one ferromagnetic layer with relaxation properties suitable for magnetic resonance imaging and detection.

13. A solution comprising a plurality of synthetic antiferromagnetic nanoparticles as set forth in claim 12, wherein said solution contains a mixture of at least two types of said synthetic antiferromagnetic nanoparticles, wherein each of said types has a distinct relaxation property and a distinct biomolecule, recognition moiety, molecular coating, or combination thereof.

14. The synthetic antiferromagnetic nanoparticle as set forth in claim 1, further comprising at least one radioactive layer.

15. A solution comprising a plurality of synthetic antiferromagnetic nanoparticles as set forth in claim 14, wherein said solution contains a mixture of at least two types of said synthetic antiferromagnetic nanoparticles, wherein each of said types has a distinct radioactive property and a distinct biomolecule, recognition moiety, molecular coating, or combination thereof.

16. The synthetic antiferromagnetic nanoparticle as set forth in claim 1, further comprising at least one dye attached to a surface of said synthetic antiferromagnetic nanoparticle.

17. The synthetic antiferromagnetic nanoparticle as set forth in claim 16, wherein said dye is fluorescent.

18. A solution comprising a plurality of synthetic antiferromagnetic nanoparticles as set forth in claim 17, wherein said solution contains a mixture of at least two types of said synthetic antiferromagnetic nanoparticles, wherein each of said types has a distinct fluorescent property and a distinct biomolecule, recognition moiety, molecular coating, or combination thereof.

19. A solution comprising a plurality of synthetic antiferromagnetic nanoparticles as set forth in claim 1, wherein said solution contains a mixture of at least two types of said synthetic antiferromagnetic nanoparticles, wherein each of said types has a distinct magnetic, optical, radioactive, or relaxation property and a distinct biomolecule, recognition moiety, molecular coating, or combination thereof.

20. A solution comprising a plurality of monodisperse synthetic antiferromagnetic nanoparticles as set forth in claim 1.
